(19) United States
(12) Patent Application Publication
(10) Pub. No.: US 2008/0243000 A1
(43) Pub. Date: Oct. 2, 2008

(54) Integrated Circuit For Implementing High-Voltage Ultrasound Functions

(75) Inventors: Benoit Dufort, Croton-On-Hudson, NY (US); Alok Govil, White Plains, NY (US); Benoit Veillette, Portland, OR (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/575,150

(22) PCT Filed: Sep. 8, 2005
(86) PCT No.: PCT/IB05/52939

§ 371 Date: Mar. 13, 2007

Related U.S. Application Data
(60) Provisional application No. 60/609,674, filed on Sep. 13, 2004.

(51) Int. Cl. A61B 8/14

(52) U.S. Cl. 600/459

(57) ABSTRACT

An integrated SOI circuit is provided for implementing high-voltage ultrasound functions of an ultrasound imaging system. The integrated circuit is packaged as an integrated chip. The integrated circuit is composed of silicon-on-insulator (SOI) technology and integrates at least the following high-voltage ultrasound functions: gatedriver, power amplifier, transmit/receive switch. Optionally the integrated chip may contain a low noise amplifier and an analog multiplexer.

The present invention relates generally to ultrasound imaging systems. More particularly, the present invention relates to an integrated circuit for implementing high-voltage ultrasound functions of an ultrasound imaging system.

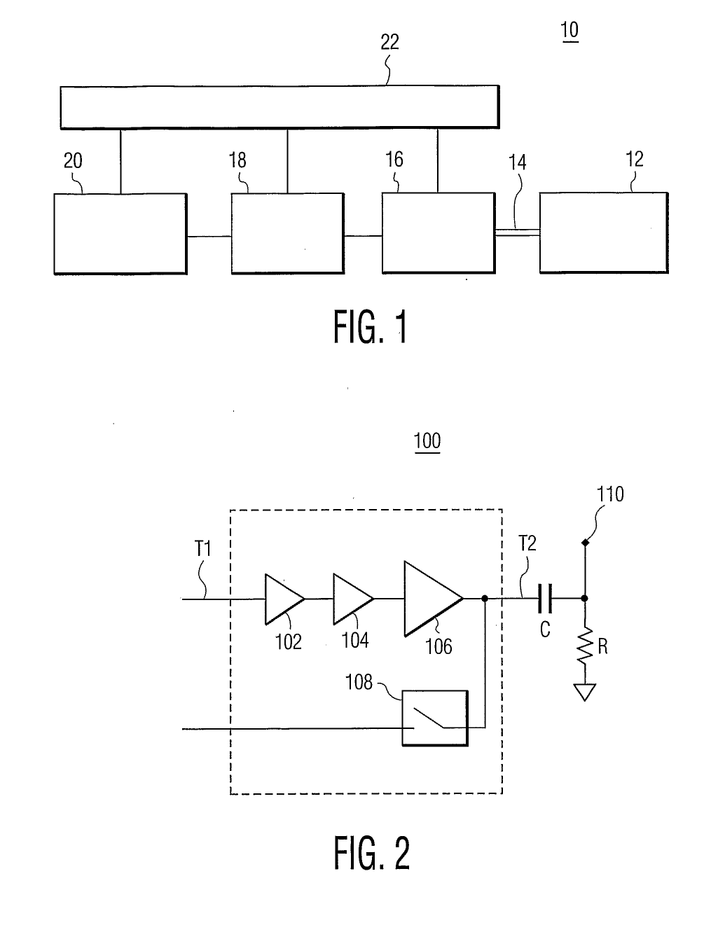

FIG. 1 illustrates an ultrasound imaging system 10. A piezoelectric transducer array 12 produces ultrasound waves from electrical stimuli and conversely translates ultrasound waves hitting it into electrical signals. The piezoelectric transducer array 12 is housed in its own casing and is linked to a cart containing the rest of the ultrasound imaging system 10 through a two-meter cable 14.

An acquisition sub-system 16 stimulates the piezoelectric transducer array 12 for the generation of an ultrasound wave. It also processes the electrical signals produced by the piezoelectric transducer array 12 from an incident ultrasound wave into a scan line. This scan line provides echogenic information about the tissues located on an axis emanating from the piezoelectric transducer array 12. A signal processing sub-system 18 converts the scan lines into an image. The image may be displayed, stored or forwarded to another system by the interface, storage and connectivity sub-system 20. A sub-system 22 provides user interface and control over the other sub-systems 16, 18 and 20.

The acquisition sub-system 16 is composed of identical channels where each deals with a single piezoelectric transducer from the piezoelectric transducer array 12. Generally, a piezoelectric transducer is used alternatively for both the generation and the reception of ultrasound waves. Each channel of the acquisition sub-system 16 thus includes a transmitter which provides a high-voltage signal to the piezoelectric transducer for the generation of an ultrasound wave, and a receiver which processes the electric signal created by an ultrasound wave absorbed by the piezoelectric transducer. A set of switches avoids the transmitter and receiver from interfering with each other.

The transmitter has the difficult task of amplifying a low-voltage, high frequency analog signal to high-voltage (typically 200 Vpp), high-current (typically +/−2 A) signal with low distortion. The transmitter includes discrete components, such as high-voltage transistors, low-voltage operational amplifiers, high-current buffers, transformers, capacitors, resistors, etc., for amplifying the low-voltage, high frequency analog signal. Accordingly, each amplifier of the acquisition sub-system 16 requires a high number of discrete components and occupies a significant board space in the system. The high number of channels in high-end ultrasound imaging systems (typically 128) results in a significant cost.

A need therefore exists for integrating the high-voltage functions of each transmitter of an ultrasound imaging system on a single integrated circuit. The single chip would greatly reduce the space required by the several discrete components of traditional ultrasound transmitter circuits which are used in achieving the high-voltage transmitter functions. Additionally, the discrete components used in traditional ultrasound transmitter circuits are optimized for other applications. Their size and power dissipation are thus higher than required. By tailoring the size and performance of all the components of the ultrasound imaging system, a single integrated circuit can offer better performance at lower power dissipation.

The present invention provides an integrated circuit for implementing high-voltage ultrasound functions of an ultrasound imaging system. The integrated circuit is packaged as an integrated chip. As such, the integrated chip of the present invention greatly reduces the space required by the several discrete components of traditional ultrasound transmitter circuits in achieving the high-voltage transmitter functions.

The integrated circuit is fabricated using silicon-on-insulator (SOI) technology. The integrated circuit is packaged as an integrated chip which integrates at least the following high-voltage ultrasound devices: gate-driver, power amplifier and transmit/receive switch for at least one channel. Optionally, a low-noise pre-amplifier and analog multiplexer can be added. The preferred SOI technology combines low-voltage CMOS technology, bipolar transistors with high-voltage, high-speed transistors that can sustain voltages in excess of ultrasound requirements on a single chip. The transistors are isolated from each other with dielectric. This results in significant area reduction versus competing technologies. It also provides for the integration on the same chip of digital logic, low-voltage and high-voltage analog functions. All these components are essential for a high-performance ultrasound transmitter.

Accordingly, the present invention integrates the building blocks of a prior art ultrasound transmitter circuit (low noise amplifier, gate driver, power amplifier, isolation diodes, T/R switches, analog multiplexer, etc.) on a single SOI chip and performs their respective functions. Moreover, the SOI integration provided by the present invention allows new functions to be added (e.g., dynamic biasing for power reduction) and new circuit techniques that are not practical or simply impossible with the several discrete components of prior art transmitter circuits.

These and other advantages will become more apparent from the following detailed description of the various embodiments of the present invention with reference to the figures wherein:

FIG. 1 is a block diagram of an ultrasound imaging system;

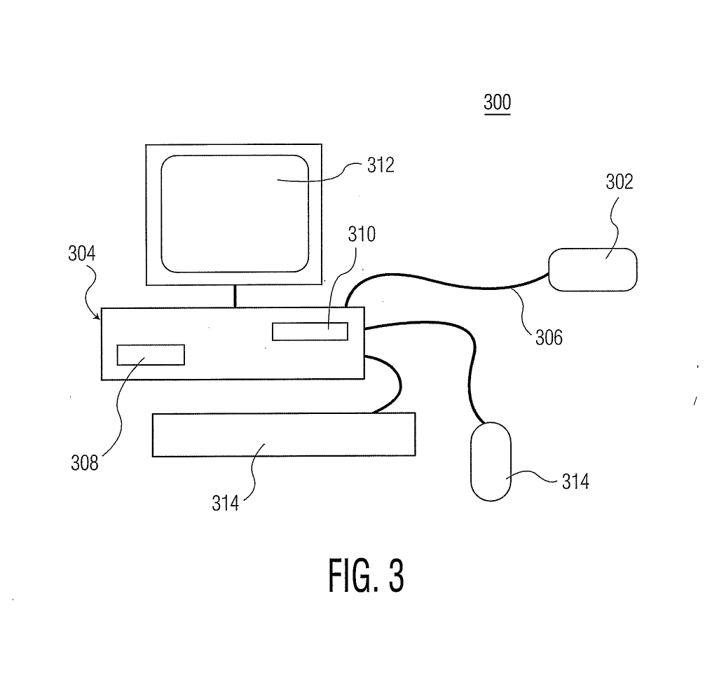

FIG. 2 is a block diagram of a single SOI integrated chip for implementing high-voltage ultrasound functions of an ultrasound imaging system in accordance with the present invention; and

FIG. 3 is a schematic diagram of an ultrasound imaging system.

Image quality in an ultrasound system depends on many factors. One of them is the number of channels available. Prior art ultrasound imaging systems are limited to 128 channels, due to power consumption and board space. The present invention provides an integrated high-voltage SOI integrated circuit for allowing more channels to be used (e.g., 256 channels) and thereby, increase image quality. The performance of a single channel is also of prime importance. Metrics such as signal-to-noise ratio, distortion and slew-rate are improved significantly with the single SOI integrated transmitter of the present invention where parasitic components are kept to a minimum.

As shown by FIG. 2, the integrated circuit is fabricated using silicon-on-insulator (SOI) technology and designated generally by reference numeral 100. The integrated circuit 100 is packaged as an integrated chip which integrates at least the following high-voltage ultrasound devices: low-noise pre-amplifier (LNA) 102, gate-driver 104 and power amplifier 106, transmit/receive switch 108. The LNA 102 can be located outside the chip 100.

The preferred SOI technology combines low-voltage CMOS technology, bipolar transistors with high-voltage, high-speed transistors that can sustain voltages in excess of ultrasound requirements on a single chip. The transistors on the SOI IC chip 100 are isolated from each other with dielectric. This results in significant area reduction versus competing technologies. It also provides for the integration on the same chip of digital logic, low-voltage and high-voltage analog functions. All these components are essential for a high-performance ultrasound transmitter.

The best way to implement the invention is to integrate as many functions of a single channel as possible onto the single chip 100, and integrate as many channels on the chip 100 as power dissipation and/or area will allow. The preferred SOI technology for the integrated circuit 100 is Philips EZ-HV, as it allows the necessary voltage range transistors (˜250 V), and offers low and medium voltage range bipolar and MOS transistors all integrated together on a single chip.

The SOI IC chip 100 takes a small amplitude analog or digital signal (low-voltage signal) signal generated by an ultrasound imaging system, such as the system shown by FIG. 1, via a first terminal (T1). The SOI IC chip 100 amplifies the low-voltage signal with a low noise amplifier (LNA) 102 for a gate driver 104, which in turns amplifies the signal for a power amplifier 106. The power amplifier 106 further amplifies the signal to output a high-voltage signal (e.g., approximately 200 Vpp) to an ultrasound probe 110 of the ultrasound imaging system (see FIG. 3) via a second terminal (T2). The ultrasound probe 110 is connected to the SOI IC chip 100 via an RC circuit having a resistor R and a capacitor C. It is contemplated that one or more of the IC chips 100 can be housed within the probe 110. The first and second terminal (T1 and T2) could be a physical pin of the SOI IC chip 100 or a signal node completely within the SOI IC chip 100.

The T/R switch 108, also on the same SOI chip 100, protects the receive electronics during transmit and offers a low impedance path to the ultrasound imaging system for at least one signal received from the ultrasound probe 108. The received signal is transmitted to the ultrasound imaging system, such as the system shown by FIG. 3.

Accordingly, the present invention integrates the building blocks of a prior art ultrasound transmitter circuit (gate driver, power amplifier, isolation diodes, T/R switches, etc.) on a single SOI chip and performs their respective functions. Moreover, the SOI integration provided by the present invention allows new functions to be added (e.g., dynamic biasing for power reduction) and new circuit techniques that are not practical or simply impossible with the several discrete components of prior art transmitter circuits. Additional circuitry can be provided within the SOI chip, such as digital logic circuitry for synthesizing the signal waveform and circuitry for converting the synthesized signal waveform to the analog domain prior to performing high-voltage amplification.

An additional embodiment of the present invention provides for an ultrasound imaging system 300 for acquiring and displaying ultrasound images, such as medical images, in order to aid in diagnosis of health related conditions as shown in FIG. 3. The ultrasound imaging system 300 includes a handheld ultrasound scanning device 302, such as an ultrasound probe, having a piezoelectric transducer array 302, a multiplexer (not shown) and at least one SOI IC chip 100 for performing high-voltage ultrasound functions as described above.

The piezoelectric transducer array 302 emits ultrasound energy in the frequency range of between 20 KHz and 20 MHz. As the ultrasound energy is reflected by tissue and structures inside a patient, the reflected energy is detected by the array 302, which in turn, relays the energy data for each channel to a control unit 304 via the multiplexer.

The control unit 304 is in electrical communication with the handheld scanning device 302 via a cable 306, which, ideally, also provides power for operation of the piezoelectric transducer array 302. Other means of communicating between the control unit 304 and the handheld scanning device 302 may be employed in addition to or in substitution of the cable 306. Such other means of communication include Bluetooth, IEEE 802.11a/b/c, infrared, etc.

The control unit 304 contains a processor 308 configured to perform a variety of image analysis and manipulation functions, and one or more storage devices 310. The storage devices 310 provide both temporary storage of the raw data received from the handheld scanning device 302 and long-term storage of processed images. The storage devices 310 may be any combination of hard drives, writeable CD-ROMs or DVDs, memory modules, magneto-optical drives and magnetic media. The control unit 304 is additionally connected to a display device 312, such as a CRT or LCD screen, for displaying the ultrasound images. Also one or more user input devices 314 are provided, allowing an operator to issue commands to the control unit 304.

It is provided that the multiplexer can be provided within the at least one SOI IC chip 100. In an alternative embodiment of the system 300, the at least one SOI IC chip 100 is located within the control unit 304. In this embodiment, the multiplexer is located within the handheld ultrasound scanning device 302.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law.

1. An integrated circuit (100) for an ultrasound imaging system (100), said integrated circuit (100) comprising:
a first terminal (T1) for receiving a low-voltage signal generated by said ultrasound imaging system (300);
means for amplifying said low-voltage signal to obtain a high-voltage signal; and
a second terminal (T2) for transmitting said high-voltage signal towards an ultrasound probe (110) of said ultrasound imaging system (300).

2. The integrated circuit (100) according to claim 1, further comprising a switch (108) for providing a low impedance path to said ultrasound imaging system (300) for at least one signal received from said ultrasound probe (110).

3. The integrated circuit (100) according to claim 1, wherein said integrated circuit (100) is fabricated using silicon-on-insulator technology.

4. The integrated circuit (100) according to claim 1, wherein said integrated circuit (100) is packaged as an integrated chip (100).

5. The integrated circuit (100) according to claim 1, wherein said means for amplifying comprises a low-noise pre-amplifier (102), a gate-driver (104) and a power amplifier (106).

6. The integrated circuit (100) according to claim 1, wherein the ultrasound probe (110) includes a piezoelectric transducer array (12).

7. The integrated circuit (100) according to claim 1, wherein the high-voltage signal is approximately 200 Vpp.

8. An ultrasound imaging system (300) comprising:
at least one integrated circuit (100) comprising:
a first terminal (T1) for receiving a low-voltage signal generated by said ultrasound imaging system (300);
means for amplifying said low-voltage signal to obtain a high-voltage signal; and
a second terminal (T2) for transmitting said high-voltage signal towards an ultrasound probe (110) of said ultrasound imaging system (300).

9. The ultrasound imaging system (300) according to claim 8, further comprising a switch (108) for providing a low impedance path to said ultrasound imaging system (300) for at least one signal received from said ultrasound probe (110).

10. The ultrasound imaging system (300) according to claim 8, wherein said integrated circuit (100) is fabricated using silicon-on-insulator technology.

11. The ultrasound imaging system (300) according to claim 8, wherein said integrated circuit (100) is packaged as an integrated chip (100).

12. The ultrasound imaging system (300) according to claim 8, wherein said means for amplifying comprises a low-noise pre-amplifier (102), a gate-driver (104) and a power amplifier (106).

13. The ultrasound imaging system (300) according to claim 8, wherein the ultrasound probe (110) includes a piezoelectric transducer array (12).

14. The ultrasound imaging system (300) according to claim 8, wherein the high-voltage signal is approximately 200 Vpp.

15. A SOI integrated chip (100) for an ultrasound imaging system (100), said SOI integrated chip (100) comprising:
a first terminal (T1);
at least one amplifier (104, 106) for amplifying a low-voltage signal received by said first terminal (T1) to obtain a high-voltage signal; and
a second terminal (T2) for transmitting said high-voltage signal towards an ultrasound probe (110) of said ultrasound imaging system (300).

16. The SOI integrated chip (100) according to claim 15, further comprising a switch (108) for providing a low impedance path to said ultrasound imaging system (300) for at least one signal received from said ultrasound probe (110).

17. The SOI integrated chip (100) according to claim 15, wherein said at least one amplifier (104, 106) comprises a gate-driver (104) and a power amplifier (106).

18. The SOI integrated chip (100) according to claim 15, wherein the ultrasound probe (110) includes a piezoelectric transducer array (12).

19. The SOI integrated chip (100) according to claim 15, wherein the high-voltage signal is approximately 200 Vpp.

20. The SOI integrated chip (100) according to claim 15, wherein the low-voltage signal is at least one of an analog and digital signal.
